(19) United States
(12) Patent Application Publication
(10) Pub. No.: US 2001/0027300 A1
(43) Pub. Date: Oct. 4, 2001

(54) Device for treatment of venous congestion

(76) Inventors: Gregory K. Hartig, Cross Plains, WI (US); Nadine P. Connor, Madison, WI (US); Michael L. Conforti, Madison, WI (US)

Correspondence Address: Keith M. Baxter, Esq., Quarles and Brady LLP, Suite 2040, 411 E. Wisconsin Avenue, Milwaukee, WI, 53202-4497, US

(21) Appl. No.: 09/745,298

(22) Filed: Dec. 20, 2000

Related U.S. Application Data
(60) Provisional application No. 60/171,351, filed on Dec. 22, 1999.

(51) Int. Cl. A61M 31/00

(52) U.S. Cl. 604/285

(57) ABSTRACT

A device for treatment of venous congestion provides for subcutaneous introduction of anticoagulant through an incision positioned within a collection shell for withdrawal of a effused material. A wash of saline and anticoagulant agitated by an air-input stream and periodic rotation of the subcutaneous device may be used to reduce clotting.

### CROSS-REFERENCE TO RELATED APPLICATIONS

This application is based on and claims the benefit of U.S. provisional application No. 60/171,351 filed Dec. 22, 1999.

### STATEMENT REGARDING FEDERALLY SPONSORED RESEARCH OR DEVELOPMENT

### BACKGROUND OF THE INVENTION

The invention relates generally to medical devices to remove excess blood from congested tissue and particularly to a simple mechanical device to replace medicinal leeches.

A potential post-surgical complication of reconstructive or microvascular surgery is venous congestion. Replanted tissue may become congested due to blood clot formation in the venous outflow of the tissue, or in any situation where arterial inflow exceeds venous outflow. Venous congestion, if not corrected by surgery or some other means, can result in tissue death.

If surgical correction fails, the current method of treating venous congestion is through the use of live medicinal leeches. The use of leeches can present a number of problems. For example, leeches can move off of congested tissue and feed on normal skin, they cannot be used near orifices of the body because of their potential for migration,. the quantity of blood removable by a leech is very limited and leeches may harbor serious pathogens. Cursory attempts have been made to develop mechanical or chemical replacements for the live medicinal leech. A simple mechanical device was used by Smoot et al. in 1995 (Smoot E. C., Ruiz-Inchaustegui J. A., Roth A. C. (1995) Mechanical Leech Therapy to Relieve Venous Congestion. J Reconstr Microsurg 11:51-55). This device consisted of a small glass bell that was placed over punch biopsy wound. A fluid pacing though an inlet port irrigated the wound and was suctioned off via a suction port at—80 mmHg. Chemical replacements for leech therapy have also been studied. The “chemical leech” involved subcutaneous injections of calcium heparin into the reattached fingers of three patients, with drainage into dressings over the surgical site. (Barnett G. R., Taylor G. I. and Mutimer K. L. (1989). The “chemical leech:” Intra-replant subcutaneous heparin as an alternative to venous anastomosis. Report of three cases. Br J Plast Surg 42:556-558. These subcutaneous injections of anticoagulant were used to promote drainage of excess blood into the dressings of the surgical site. However, prior work has not provided an adequate clinical solution for the post-surgical complication of venous congestion. The need for the development of new techniques is clearly indicated.

### SUMMARY OF THE INVENTION

The present invention provides an improved device for the treatment of venous congestion. In one non-limiting embodiment, the device consists of a glass shell, which acts as a collection chamber and supports several additional components of the device. These components include a means to 1) supply anticoagulant subcutaneously through a skin incision, 2) supply anticoagulant to the surface of the incision, 3) apply turbulence to the surface of the incision, and 4) supply anticoagulant to the peripheral tissue surrounding the incision. Specifically, the invention provides a shell with a rim adapted to be affixed to the patient's skin defining a suction area circumscribed by the rim and inner volume of the device. A conduit supported by the shell has a delivery tip placed subcutaneously through a skin incision for the delivery of anticoagulant. This subcutaneous delivery tip may be made out of a porous material such as a microporous polyethylene impregnated with a polyvinyl alcohol hydrogel or possibly hypodermic stainless steel tubing configured into a semispherical wireframe with pinholes spaced along the tubing to allow for anticoagulant egress. The purpose of this delivery tip is to 1) supply concentrated anticoagulant subcutaneously in a controlled fashion, 2) to provide mechanical anticoagulation by automated rotational movement of the delivery tip, 3) to provide subcutaneous tenting so as to keep open (apart) the skin incision edges. Suction is applied to the glass shell via an outflow port allowing recovered blood and anticoagulant to be withdrawn from the inner chamber.

It is one object of the invention to provide for improved removal of blood from congested tissue through the combination of subcutaneous delivery of anticoagulant and topical recovery.

Another object of the invention is to provide for improved dispersal of anticoagulants subcutaneously and to allow rotary motion of the tip so as to inhibit clotting. Besides subcutaneous anticoagulant delivery via the delivery tip, additional anticoagulant can be delivered via needle injection around the shell rim at equidistant intervals. This subcutaneous anticoagulant will be delivered peripheral to the skin incision.

The device may include an inlet port allowing the introduction of an irrigant such as an anticoagulant-saline mixture into the inner volume. The inlet port may carry irrigant to the skin surface. Thus it is another object of the invention to improve blood removal by augmenting the subcutaneous delivery of anticoagulant with a topical irrigant.

The device may include an air inlet port allowing the introduction of air into the inner volume and down to the skin surface. Thus it is another object of the invention to both provide a path of air entry to the skin surface. This air flow will create turbulence in the irrigant flowing through the shell at the skin surface, thus creating mechanical anticoagulation at the skin surface and elsewhere within the shell preventing clot formation.

The device may include a sensor detecting blood outflow concentration such as an optical sensor.

Thus it is another object of the invention to provide for semiautomatic operation in which a sensor provides an indication to the operator of successful operation or triggers sequences of agitations and air and liquid flows to provide for efficient blood removal.

The foregoing objects and advantages may not apply to all embodiments of the inventions and are not intended to define the scope of the invention, for which purpose claims are provided. In the following description, reference is made to the accompanying drawings, which form a part hereof, and in which there is shown by way of illustration, a preferred embodiment of the invention. Such embodiment also does not define the scope of the invention and reference must be made therefore to the claims for this purpose.

### BRIEF DESCRIPTION OF THE DRAWINGS

FIG. 1 is an exploded perspective view of the device of the present invention showing its disassembly prior to insertion of a subcutaneous conduit into a cross incision in the patient's skin and the placement of a collection shell over the conduit, and prior to attachment with various input lines and outflow lines;

FIG. 2 is an elevational cross sectional view of the device of FIG. 1 assembled and attached to the patient's skin and showing the subcutaneous location of the delivery tip of the conduit formed from a microporous disk and showing the placement of air and irrigation tubes and a suction port on and in the collection shell;

FIG. 3 is a fragmentary cross-sectional view similar to that of FIG. 2 showing an alternative embodiment wherein the subcutaneous conduit is attached to a motor for automatic periodic motion;

FIG. 4 is a fragmentary view of FIG. 2 showing the use of an optical sensor for detecting blood outflow such as may be used to control various aspects of the invention; and

FIG. 5 is a perspective view similar to that of FIG. 1 showing the addition of a series of needles positioned within the rim of the collection shell for injecting additional anticoagulant around the shell rim at predetermined intervals

### DETAILED DESCRIPTION OF THE INVENTION

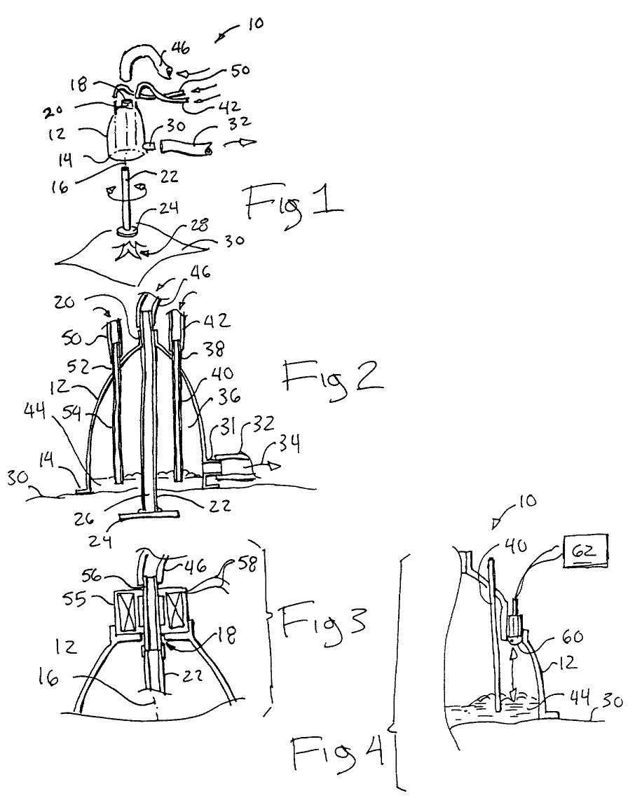

Referring now to FIG. 1, the device 10 of the present invention includes generally a hollow, bell-shaped shell 12 symmetric generally about vertical axis 16 and having an open lower rim 14. The shell 12 may be constructed of plastic or glass and is preferably of clear material to allow visual inspection of its internal volume.

At the apex of the shell 12 is an opening 18 surrounded by a cylindrical sleeve 20. The sleeve 20 is sized to receive along axis 16, a conduit 22, the latter being preferably a stainless steel tube having a height greater than that of the shell 12. The conduit 22 may freely rotate within the sleeve 20 but nevertheless blocks the opening 18 so as to prevent passage of air or liquid into or out of the opening 18 except through the conduit 22.

Referring now also to FIG. 2, attached at a lower end of conduit 22 removed from the sleeve 20 is a microporous disk 24 having an internal structure of pores (not shown) communicating with a central lumen 26 of the conduit 22. The disk 24 is centered on the conduit 22 extending radially therefrom generally perpendicular to axis 16.

A cross incision 28 made in the skin 30 of a patient permits insertion of the disk 24 subcutaneously with the conduit 22 extending upward out of the incision 28. The portion of the conduit 22 extending out of the incision 28 is received by the sleeve 20 so that the shell 12 moves downward to abut the skin 30 and cover the cross incision 28. The diameter of the rim 14 of the shell 12, in the preferred embodiment, is approximately 1.3 centimeters.

The conduit 22 may be attached at its upper end protruding from the sleeve 20 to a anticoagulant supply hose 46 delivering concentrated Heparin through the conduit 22 into the microporous disk 24 for diffusion subcutaneously in the surrounding area.

Extending radially near the rim 14 of the shell 12 outside of the shell 12 is an exhaust port 31 sized to receive a suction hose 32 and providing an exhaust path indicated by arrow 34 in FIG. 2 from an inner volume 36 of the shell 12 (defined by the inner walls of the shell 12 and the upper surface of the skin 30) to the suction hose 32. The exhaust port 31 is positioned to draw effluent liquid 44 collecting on the upper surface of the skin 30 out of the shell 12.

An air inlet port 38 extends vertically upward from a top of the shell 12 to receive an air supply hose 42 and to communicate air therefrom through the shell 12 to a central air tube 40 extending downward within the shell to a point immediately above the surface of the skin 30. Ideally the opening of the tube 40 is slightly below the opening of the exhaust port 31 so as to ensure the tip of the air inlet port 38 is immersed in any unexhausted effluent liquid 44.

Similarly, an irrigation port 52 extends vertically upward from a top of the shell 12 opposed to the air inlet port 38 about the sleeve 20 to receive an irrigation hose 50 and to communicate irrigation liquid therefrom through the shell 12 to an irrigation tube 54 similar to the air tube 40 extending downward within the shell to a point immediately above the surface of the skin 30. Tubes 40 and 54 may be stainless steel hypodermic needle tubes.

Referring still to FIGS. 1 and 2, in operation, the disk 24 is first vacuum impregnated heparin polyvinyl alcohol hydrogel and implanted in the tissue through the cross incision 28 described above. The shell 12 is then be placed over the conduit 22, the latter fitting through sleeve 20, and positioned to cover the incision 28 with the rim 14 resting on the surrounding skin. The rim 14 of the shell 12 is attached to the skin 30 using a surgical adhesive or an outer flange extension on the shell 12 may be captured beneath the specially designed adhesive strip in the form of an annular ring.

Hose 46 is then attached to the portion of the conduit 22 extending out of the shell 12 through sleeve 20, while hoses 32, 42 and 50 may be pre-attached to the shell 12.

Concentrated Heparin is next delivered through the conduit 22 into the microporous disk 24 for diffusion subcutaneously in the surrounding area. Encouraged by the anticoagulant, blood in the region of the disk 24 is drawn up through the incision 28. The extracted blood and anticoagulant then mixes with the irrigant introduced through tube 54. The irrigant is preferably a wash of dilute anticoagulant and saline solution and serves to further inhibit the formation of clots in the resulting effluent liquid 44.

Air entering through an air inlet hose 42 through the tube 40 percolates air bubbles through effluent liquid 44, the bubbles serving further to inhibit the formation of clots on the incision surface. Pulsations of pressure, air and irrigant may also be used to improve blood flow.

Periodically, the conduit 22 is rotated in alternate directions so as to reduce the formation of clots around the disk 24. The disk shape and its orientation perpendicular to the axis of rotation facilitate this rotational process.

Anticoagulant, irrigation, airflow and suction are balanced to establish a slight negative pressure within the shell 12 with respect to ambient pressure. The delivery of air, saline and anticoagulant and the application of suction may be performed by an automated control system comprising pumps and pressure transducers and a programmed controller according to techniques well known in the art.

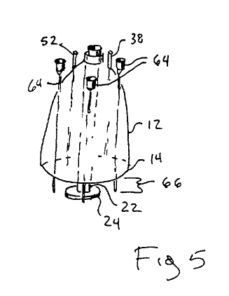

Referring now to FIG. 3 in an alternative embodiment, a stepper motor 55 may be positioned at the apex of the shell 12 so that its shaft 56 is essentially coaxial with axis 16 and conduit 22. The shaft 56 may be hollow so as to permit passage of anticoagulant therethrough and the lower portion of the shaft may extend through the opening 18 to be attached to the conduit 22. The opposite, upper end of the shaft 56 may be attached to hose 46. Signals received through motor wires 58 from an automatic controller of a type well known in the art may drive the motor to produce a periodic reciprocating motion of the shaft 22 to eliminate the need for manual intervention.

Referring now to FIG. 4, an optical sensor 60 may be fit within the wall of the shell 12 to detect color changes in the effluent liquid 44 collecting in the lower portion of the shell adjacent to the skin 30. Ideally the sensor 60 is placed near the exhaust port 31 (not shown in FIG. 4) and may include, for example, a light emitter (such as a light emitting diode) and light detector (such as a photo transistor) for evaluating the color or reflectance of the effluent liquid 44. This measurement may be used to indicate the amount of blood outflow so as to provide a signal through a controller 62 either to attending personnel that rotation of the conduit 22 is required, or an inspection of the device is required, or to automatically actuate changes in the air flow, irrigation flow, or mechanical agitate the conduit through the motor shown in FIG. 3.

Referring now to FIG. 5 in an additional embodiment, the shell 12 may support a set of vertically disposed hypodermic needles 64 generally parallel to the conduit 22 and spaced at regular angular intervals about the conduit 22 just inside the rim 14 and extending a distance 64 below the rim 14 to provide for the injection of additional anticoagulant subcutaneously around the disk 22.

It is specifically intended that the present invention not be limited to the embodiments and illustrations contained herein, but that modified forms of those embodiments including portions of the embodiments and combinations of elements of different embodiments also be included as come within the scope of the following claims.

We claim:

1. A device for the treatment of venous congestion comprising:
a shell having a rim adapted to abut a patent's skin to define a suction area circumscribed by the rim and an inner volume;
a conduit supported by the shell and having a delivery tip positionable subcutaneously below the rim within the suction area when the shell is positioned against the patient's skin, for the delivery of anticoagulant; and
a suction port attached to the shell through which recovered anticoagulant may be drawn from the inner volume.

2. The device of claim 1 wherein the delivery tip includes a diffusion material for dispersing the flow of anticoagulant.

3. The device of claim 2 wherein the diffusion material is a polyethylene microporous material.

4. The device of claim 2 wherein the diffusion material is a disk extending substantially parallel to a plane of the patient's skin.

5. The device of claim 1 wherein the conduit extends normal to the patient's skin.

6. The device of claim 1 wherein the conduit is supported by the shell to permit axis rotation of the conduit.

7. The device of claim 1 including a rotational actuator for providing a rotation to the conduit.

8. The device of claim 7 wherein the rotation actuator provides for a predetermined reciprocation of the conduit.

9. The device of claim 1 including further a wash inlet port allowing the introduction of a wash liquid into the inner volume.

10. The device of claim 9 wherein the wash inlet port communicates with a tube positioned in the inner volume and communicating introduced wash liquid to a region proximate to the patient's skin.

11. The device of claim 1 wherein the wash inlet port includes a fitting external to the shell for attachment to a source of pressurized wash liquid.

12. The device of claim 1 including further an air inlet port allowing the introduction of air into the inner volume.

13. The device of claim 12 wherein the air inlet port communicates with a tube positioned in the inner volume and communicating introduced air to a region proximate to the patient's skin.

14. The device of claim 12 wherein the air inlet port includes a fitting external to the shell for attachment to a source of pressurized air.

15. The device of claim 1 including further a sensor detecting blood outflow.

16. The device of claim 1 wherein the sensor is attached to operator indicator.

17. The device of claim 1 providing at least one needle for injecting additional anticoagulant around the shell rim at predetermined intervals.

18. A method for the treatment of venous congestion comprising:
(a) implanting through an incision in the skin, a tip of an anticoagulant delivery conduit;
(b) placing a shell over the incision, the shell having a rim surrounding the incision on the patient's skin to define an inner volume, the shell further having a suction port communicating with the inner volume;
(c) delivering an anticoagulant through the delivery conduit; and
(d) withdrawing recovered blood and anticoagulant through the suction port.

19. The method of claim 18 wherein the tip includes a diffusion material for dispersing the flow of anticoagulant and including the step of impregnating the diffusion material with anticoagulant prior to implantation.

20. The method of claim 19 wherein the anticoagulant is a mixture of Heparin and polyvinyl alcohol.

21. The device of claim 18 wherein the conduit is supported by the shell to permit axis rotation of the conduit and including the step of periodically rotating the conduit to prevent the formation of clots.

22. The method of claim 18 wherein the rotation reciprocates of the conduit.

23. The method of claim 18 including wherein the shell includes a wash inlet port allowing the introduction of air into the inner volume and including the step of washing the surface of the skin near the incision with an anticoagulant.

24. The method of claim 18 including wherein the shell includes an air inlet port allowing the introduction of air into the inner volume and including the step of percolating air through liquid material adjacent to the skin to cause an agitation of that liquid.
